(19) United States
(12) Patent Application Publication
(10) Pub. No.: US 2026/0301283 A1
(43) Pub. Date: Oct. 1, 2026

(54) COMPUTER-IMPLEMENTED MAA METHOD FOR OPERATING AN X-RAY FACILITY, X-RAY FACILITY, COMPUTER PROGRAM AND ELECTRONICALLY READABLE DATA CARRIER

(71) Applicant: Siemens Healthineers AG, Forchheim (DE)

(72) Inventors: Maximilian Rohleder, Erlangen (DE); Björn Kreher, Bräuningshof (DE)

(21) Appl. No.: 19/632,287

(22) Filed: Mar. 28, 2026

(30) Foreign Application Priority Data
Mar. 28, 2025 (EP) 25167054

(51) Int. Cl. G06T 12/30

(52) U.S. Cl. CPC G06T 12/30; G06T 2207/30052

(57) ABSTRACT

A method for operating an X-ray facility is provided. Before the recording of a recording region that includes at least one metal object relevant to artifact formation, an object information item that defines the three-dimensional position, orientation, and extent for each metal object relevant to artifact formation is determined. From the object information item for each metal object relevant to artifact formation, an object model is determined that defines at least one three-dimensional basic geometric form that defines the metal object relevant to artifact formation and its position, size and orientation in the recording region. An artifact measurement information item that defines the strength of metal artifacts that occur, for one or more metal objects relevant to artifact formation and at least one projection geometry making use of one or more object models, is determined. The artifact measurement information item is used in determining the artifact-reducing recording trajectory.

This application claims the benefit of European Patent Application No. EP 25167054, filed on Mar. 28, 2025, which is hereby incorporated by reference in its entirety.

### BACKGROUND

The present embodiments relate to operating an X-ray facility that includes a recording arrangement with an X-ray radiator and an X-ray detector.

It is known in the prior art to perform computed tomography, inter alia, with non-dedicated X-ray facilities. For example, it is known, in an X-ray facility with a C-arm, to realize the recording trajectory for recording two-dimensional projection images using the C-arm on which the X-ray radiator and the X-ray detector are arranged opposite one another. Since, in such processes, it is usually a cone beam geometry of the beam field that is used, it is also cone beam computed tomography (CBCT) that is considered here. By way of example, image recordings may take place for preparation, for support, and/or for subsequent evaluation and for medical interventions.

One problem is artifacts caused by metal objects in the recording region. This is particularly relevant if the metal objects themselves and/or immediately adjacent anatomical regions are the subject and/or target of the imaging. For example, it is known, when placing implants, to check the positioning of the implants by way of CBCT. A specific example is the placement of pedicle screws. In such applications, metal artifacts obscure clinically relevant anatomical details close to the metal object so that the clinical usefulness of CBCT image datasets also becomes reduced.

For this reason, it has been proposed in the prior art to apply methods for reducing metal artifacts in the post-processing (e.g., metal artifact reduction (MAR)) and/or for avoiding severe metal artifacts during data recording (metal artifact avoidance, MAA).

By way of example, for MAR, inpainting methods are known in which firstly voxels containing metal (e.g., metal voxels) are segmented in a first reconstruction, whereupon the projection data in the projection line (e.g., the ray course) that contributes to the metal voxel is processed in order to remove the contribution of the metal for the reconstruction. In cases of severe artifacts, however, the deformation of the metal objects leads to an oversegmentation or undersegmentation of the metal objects in the first reconstruction, so that the effectiveness of such algorithms is limited for severe artifacts.

In MAA methods, efforts are made to improve the quality of the raw data recorded (e.g., the projection images) by adapting the recording trajectory during the image recording. Since projection images with a large metal bias are avoided, three-dimensional image datasets that are reconstructed from this more reliable projection data have fewer metal artifacts and, for example, no excessively severe metal artifacts. Since the optimized recording trajectory depends upon the position, shape, and orientation of the metal objects, additional X-ray images (e.g., scout images) are needed in order to predict an artifact-reducing (e.g., artifact-avoiding) recording trajectory for the scene to be recorded. It is herein known, for example, following the recording of the scout images, to establish therefrom a three-dimensional metal mask that maps the voxels containing metal in order then to simulate projection images of all the possible projection geometries and thereby to determine the best recording trajectory such that the metal path (e.g., metal transirradiation length) is minimized. MAA methods are described, for example, in US 11,790,525 B2.

It is noted in an article by Maximilian Rohleder et al., “An interactive task-based method for the avoidance of metal artifacts in CBCT,” International Journal of Computer Assisted Radiology and Surgery (2024) 19:1399-1407, that MAA methods that determine an artifact-reducing trajectory are still difficult to use due to remaining methodological limitations and a lack of workflow integration in clinical practice. It is therefore proposed to detect and visualize the spatial distribution and the calibrated strengths of expected artifacts for a given tilted circular recording trajectory so that a user may select an optimum recording trajectory interactively.

A substantial problem in existing MAA methods that propose an artifact-reducing recording trajectory based on a metal mask is that the recording trajectory is adapted for all the metal voxels of the recording region, which may lead, for example, to irrelevant objects exerting an influence on the selection of the recording trajectory or even being falsely prioritized in contrast to clinically relevant metal objects such as implants, so that sub-optimal recording trajectories may result. In this regard, in the above-mentioned article by Rohleder et al., it is proposed to determine local artifact distributions and to permit clinicians to prioritize regions of interest. A further existing problem is that when metal masks are used in the determination of the optimized trajectory, complex calculations and/or simulations are often necessary (e.g., including forward projection and/or rendering with regard to the complex three-dimensional metal mask), which result in longer computation times.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, an improved, easily usable possibility that may be realized and/or improved in a computationally efficient way, aimed at the imaging goal of reducing (e.g., avoiding) metal artifacts during a recording procedure is provided.

In a method according to the present embodiments, for operating an X-ray facility that includes, for recording two-dimensional projection images in different projection geometries along a recording trajectory, from which a three-dimensional image dataset may be reconstructed, a recording arrangement with an X-ray radiator and an X-ray detector, it is provided according to the present embodiments that before the recording of a recording region that includes at least one metal object relevant to artifact formation, for determining an artifact-reducing recording trajectory: an object information item that defines the three-dimensional position, orientation, and extent for each of the at least one metal object relevant to artifact formation is determined; from the object information item for each metal object relevant to artifact formation, an object model is determined that defines at least one three-dimensional basic geometric form that defines the metal object relevant to artifact formation and its position, size, and orientation in the recording region; and an artifact measurement information item that defines the strength of metal artifacts that occur, for at least one of the at least one metal object relevant to artifact formation and at least one projection geometry (e.g., at least one potential recording trajectory), making use of at least one of the at least one object model. The artifact measurement information item is used in determining the artifact-reducing recording trajectory.

The method is suitable, for example, for cone beam computed tomography (CBCT) (e.g., with a C-arm that is able to provide a large number of degrees of freedom with regard to the recording trajectory). In a specific embodiment, for example, it may be provided that the artifact-reducing recording trajectory is determined as a circular trajectory tilted by a tilt angle capable of being optimized. In the case of a C-arm, it may be tilted, for example, by the tilt angle in order to adjust the adapted artifact-reducing recording trajectory. In developments, however, more complex artifact-reducing recording trajectories that deviate from circular paths may be realized in that the trajectory parameters to be optimized are selected accordingly. Reconstruction techniques for determining three-dimensional image datasets with non-circular recording trajectories are already known in the prior art.

In first embodiment variants, all metal objects situated in the recording region and the reconstruction region reconstructed in the three-dimensional image dataset in the vicinity of which metal artifacts may form may be understood to be of relevance to artifact formation (e.g., all metal objects in the reconstruction region). In one embodiment, as described in greater detail below, objects relevant to artifact formation in second embodiment variants may, however, also be formed by a subset of all the metal objects present. In one embodiment, metal objects that belong to an object class that is to be represented as an imaging target and/or that lie within a region of interest that is to be represented as an imaging target as free as possible from artifacts are relevant to artifact formation.

The approach set out for determining an artifact-reducing recording trajectory based on artifact measurement information is, terminologically, an MAA method (e.g., a method for metal artifact avoidance); although, in general, residual artifacts may remain, and complete avoidance is typically not possible. The artifact avoidance therefore relates to metal artifacts of a pre-determined strength and/or type that, for example, severely impede and/or render impossible the interpretation of the image dataset formed and therefore counteract the imaging aim. Since, in general, no complete avoidance of artifacts is possible, the designation “artifact-reducing recording trajectory” has been chosen. The artifact reduction (e.g., and avoidance) relates to one or more of the at least one metal object relevant to artifact formation, and this is considered in greater detail below.

According to the present embodiments, the evaluating function may be used to detect metal objects relevant to artifact formation in a targeted manner and to determine properties of the metal objects in order to be able to operate with object models based on a simple geometric basic form rather than an abstract metal mask. Therefore, initially, metal objects relevant to artifact formation are detected, and at least one property of each metal object is determined. This is defined by the object information item. This is suitable for parameterizing the at least one basic geometric form that is to be used for the metal object relevant to artifact formation so that a clear position, orientation, and extent of the basic geometric form results as the object model. From this at least one object model, it may now be estimated how strongly at least one projection geometry contributes to metal artifacts and/or, for example, for a complete recording trajectory, how severe the metal artifacts arising are likely to become. This provides that an artifact measurement information item is determined that describes the pre-calculated artifact strength of metal artifacts arising during the reconstruction of a three-dimensional image dataset. This may be profitably used in different ways to determine an artifact-reducing recording trajectory that is to be used. These variants have already been described, in principle, for metal masks and may also be used cumulatively.

There are a number of possibilities for specific determination of the object information item. First, it may be provided that the object information item is determined at least partially from navigation data of at least one navigation system for support during a preceding intervention for positioning the metal object relevant to artifact formation. This is suitable, for example, if the X-ray facility (e.g., a C-arm X-ray facility) is part of an intervention workplace for performing interventions (e.g., medical interventions) on an examination object (e.g., a patient). The intervention workplace may then also have a navigation system that tracks the position of particular objects relevant to the intervention (e.g., also the metal objects relevant to artifact formation and/or instruments and/or materials used for positioning the metal objects relevant to artifact formation). Navigation systems of this type, which are also known as tracking systems and/or position-determining systems, have previously been described in the prior art. Their navigation data may describe (e.g., during the tracking described above of metal objects relevant to artifact formation and/or of instruments and/or materials used for positioning the metal objects relevant to artifact formation) the position and/or orientation of metal objects relevant to artifact formation in a coordinate system of the navigation system that is registered to that of the X-ray facility. In this regard, it may be suitable if at least one prior information item describing the form and extent of the metal object is also used (e.g., in relation to the extent). Thus, in embodiments, a determination of the object information item is possible, for example, without the recording of two-dimensional scout images with the X-ray facility.

Additionally or alternatively, it may be provided that at least one two-dimensional scout image of the recording region is recorded, and the at least one scout image is evaluated using at least one evaluating function to determine the object information item that describes at least the position, orientation, and extent for each of the at least one metal object relevant to artifact formation in one of the at least one scout images. Often, such scout images are also used for other purposes so that their recording often involves no actual extra effort. Possibly in addition to navigation data, or independently thereof, they contain valuable indications of the position, orientation, and possibly extent of the metal objects relevant to artifact formation that may be obtained via a (e.g., trained) evaluating function that is applied to the at least one scout image.

In this regard, it may be suitable if at least two two-dimensional scout images (1) of the recording region are recorded in different (e.g., mutually perpendicular projection geometries) and evaluated. The three-dimensional position, orientation, and extent for each of the at least one metal object relevant to artifact formation is determined at least partially from the scout image-related object information and the projection geometries of the scout images. For example, reference points that have been determined via the evaluating function in a plurality of two-dimensional scout images may also be located three-dimensionally by triangulation when the projection geometries are known. However, example embodiments may also be provided in which a single two-dimensional scout image is sufficient if at least one prior information item that describes the form and extent of the metal object is used for determining the three-dimensional position, orientation, and extent for each of the at least one metal object relevant to artifact formation. Often, more accurate prior information regarding metal objects relevant to artifact formation situated in the recording region is available. If it describes the form and extent sufficiently accurately, from the mapping in a single scout image, in many cases, it may already be concluded where in space the metal object relevant to artifact formation is situated and how it is oriented. Such prior information is also suitable in the case of a plurality of scout images for more exact determination of the object information and also with navigation data.

It may be provided that the artifact-reducing recording trajectory and/or at least one suggestion set of optimized recording trajectories that is offered to a user for selection is determined in an optimizing process minimizing a target function. The target function includes at least one artifact measurement term determined from the artifact measurement information item. This provides that the determination of the artifact-reducing recording trajectory may be based centrally upon an optimization process running automatically on the control facility of the X-ray facility. Therein, a single optimized recording trajectory may be determined as an artifact-reducing recording trajectory to be used; although it is also conceivable to output a plurality of optimized recording trajectories (e.g., a number of the best results) and to leave the final selection to the user.

Further, when the artifact measurement information item is used, it may be provided that the artifact measurement information item is determined in a metal object-specific manner and is used for object-specific visualization of an expected artifact strength per metal object relevant to artifact formation in a representation that serves for user selection of an artifact-reducing recording trajectory to be used and/or for the user selection of at least one of the at least one metal object relevant to artifact formation. Therefore, if an artifact measurement information item resolved by metal objects is provided to the user regarding how strongly and at what recording trajectory which metal object relevant to artifact formation contributes to metal artifacts (e.g., for the plurality of optimized recording trajectories of the optimization process), he/she may make a well-founded decision for a recording trajectory and/or possibly a recording trajectory portion and/or projection geometries to be used that may be oriented to the imaging target. It is also conceivable, through the selection of particular metal objects relevant to artifact formation, to focus the determination of the artifact-reducing recording trajectory (e.g., the optimization process) thereon, in order to achieve, for example, an optimized imaging quality for the region around these selected metal objects relevant to artifact formation. For example, it may also be provided that on user selection of one of the at least one metal object relevant to artifact formation, the optimization process relating to the selected metal object relevant to artifact formation is carried out. Underlying this is the concept that metal artifacts form, for example, around the metal object triggering them. If a metal object and/or the anatomy round the metal object is to be seen particularly clearly, it is therefore useful to select this metal object relevant to artifact formation and therefore to prioritize it and/or consider it alone. With regard to this metal object of interest relevant to artifact formation, an optimization may be achieved and/or an artifact-reducing recording trajectory that optimizes the image quality for it may be selected in that metal artifacts are avoided as far as possible. The display of the artifact measurement information item may take place, for example, via coloring the respective metal objects relevant to artifact formation in a representation that is abstracted and/or based upon a scout image.

In the optimization process (e.g., as a term of the target function and/or as at least one boundary condition), the evaluation capability of a resulting three-dimensional image dataset may be taken into account with regard to an imaging aim and/or the technical feasibility of the recording trajectory with the X-ray facility. Generally speaking, different further optimization goals may be incorporated and may also be specific for an examination procedure (e.g., the arrangement of different components about a patient to be imaged). For example, such further terms of the target function and/or boundary conditions are directed to the fundamental technical feasibility of the artifact-reduced recording trajectory and/or the best possible image quality with regard to the imaging goal. However, optimization with regard to comfort may also be carried out, for example, in the context of a medical intervention by avoiding the need to clear areas of components and persons.

Generally speaking, therefore, according to the present embodiments, as distinct from existing approaches, it is provided that metal objects in a scene are explicitly detected and parameterized rather than estimating metallic voxels as a three-dimensional metal mask without taking account of individual metal objects. This approach may therefore be designated a parametric MAA (P-MAA). The modeling of metal objects relevant to artifact formation via basic geometric forms permits a clear reduction in computation times for the artifact measurement information item, as investigations have shown. This applies, for example, also, if images are rendered, for example, for transirradiation lengths since then also the computation times may be reduced, for example, by a factor of four to six. For analytical approaches still to be discussed in detail, a significantly greater reduction in the computation effort may be achieved, for example, by a factor or twenty to forty. Stated differently, the present embodiments permit the previously extremely time-consuming and resource-consuming MAA process to be accelerated significantly and the effort to be significantly reduced. Where, previously, projection images of the three-dimensional metal mask were amended, even if implemented on a GPU using ray tracing, this may take a number of minutes. In the method described here, however, metal objects relevant to artifact formation are modeled via basic geometric forms so that in a compact manner, the position, orientation, and extent of each metal object relevant to artifact formation may be mapped in the object model. This enables a significant acceleration of the determination of the artifact measurement information item, which, however, may be determined in sufficiently good quality in order to be able to determine artifact-reducing recording trajectories automatically and/or with user assistance.

In order to determine the artifact measurement information item, a metal transirradiation length may suitably be determined for one or more of the at least one metal object relevant to artifact formation. In the prior art, a plurality of predictors for the strength of metal artifacts have previously been proposed, which are typically all based upon metal transirradiation lengths. These methods for determining artifact measurement information that may also be used in the present embodiments include, for example, the spectral shift method, the scatter-to-total ratio method, and the total attenuation. Specifically, it may be provided that the metal transirradiation length is achieved by rendering a transirradiation length image for the at least one projection geometry for one or more of the at least one object model, as previously stated. With such an approach to image synthesis, significant reductions in the computation effort (e.g., the computation times) may already be achieved. In one embodiment, however, it may be provided that the calculation of the metal transirradiation length is achieved using at least one analytical relationship based upon a property of the basic geometric form. When an analytical relationship is used, no determination of a transirradiation length image, no calculation of a plurality of X-ray paths, and no, for example, complex simulation may be provided or needed, resulting in a significant reduction of the computation effort and opening up MAA methods for use in clinical practice in further improved ways. Further, the use of a graphics processing unit (GPU) may be dispensed with, and central processing units (CPUs) alone may be utilized.

Suitably, the “total attenuation” approach is selected since it is a simple and efficient predictor of metal artifacts (e.g., those caused by photon starvation that is critical in medical interventions for the insertion of metallic implants, such as pedicle screws). In the specific example of pedicle screws, for example, the phenomenon of screw shaft blooming is known, in which the pedicle screws appear larger than their actual size. This may make the checking of their placement in the pedicle canal more difficult. In the “total attenuation” method, the degree of metal artifacts is assessed by judging the maximum cross-over of X-ray beams and metal objects.

In order to use “total attenuation,” it may therefore be provided that a maximum transirradiated metal path, therefore the maximum metal transirradiation length, is determined via the at least one object model. Then, for many basic geometric forms, an analytical relationship may be derived that enables it to be stated directly without rendering for a transirradiation direction (e.g., by projection geometry) how great the maximum metal transirradiation length is. However, analytical relationships may also be determined for other metal transirradiation lengths. For example, the basic form is selected such that the longest transirradiation length through the object model results directly mathematically from any desired projection directions relative to the basic form in the analytical relationship. In this way, the number of calculations needed is proportional only to the number of basic geometric forms so that the computation time and the computation effort are significantly reduced.

In an optimization process, it may be provided, for example, that the artifact measurement term is a transirradiation term that describes the maximum transirradiated metal path through the at least one object model for the projection images of the respective recording trajectory.

Even in an analytical consideration of transirradiation lengths via individual object models, account may be taken of a plurality of metal objects relevant to artifact formation that are potentially transirradiated along an X-ray beam, with little effort, for example. It is checked whether the object models lie in a line, and/or for example, it is checked whether a main axis of a basic form intersects another basic form, what proportion may then be added in this direction. For many applications, however, an overlap along X-ray beams may also be ignored. For example, in the case of pedicle screws, the longitudinal axes may be arranged sufficiently far removed and/or transversely offset from one another. Given typical screw dimensions, an overlap in relation to a transverse axis contributes less to the photon starvation since the diameters are significantly smaller than the lengths.

In one embodiment, it may be provided that, as the basic geometric form, an ellipsoid is used. Ellipsoids have proved to be favorable, not only with regard to a potential rendering, for example, of transirradiation length images in the calculations, but also with regard to the use of an analytical relationship. It may, for example, be provided that the maximum metal transirradiation length for an object model is calculated extending through the geometric center of the basic form (e.g., an ellipsoid) of the respective object model. Therefore, use is made of the insight that the maximum projection of an ellipsoid always extends through its geometric center (e.g., centroid), which permits an efficient calculation of the artifact measurement information item without the need for rendering. For example, an extremely drastic reduction of the calculation times (e.g., by a factor of 33) is possible.

Alternatively and/or additionally to an ellipsoid, other basic geometric forms may be provided (e.g., cuboids (for plate-like implants), cylinders, and suchlike). Here also, analytical relationships may be determined, as may be shown. Ellipsoids as a basic form are suitable, for example, for elongate metal objects relevant to artifact formation, such as screws, Kirschner wires (K-wires), and suchlike. In general, it may be provided that one or more of the at least one metal object relevant to artifact formation is a screw implant (e.g., a pedicle screw).

Cases may also be provided in which individual basic geometric forms are less suitable for mapping metal objects relevant to artifact formation. In such a case, in a development of the present embodiment, one or more of the at least one metal object relevant to artifact formation may be modeled in the object model via a plurality of overlaid basic forms. In this way, more complex forms of metal objects may be modeled. For example, a few basic forms (e.g., a few ellipsoids) are used for simulating the more complex form.

As previously mentioned, the evaluating function that is applied to the at least one scout image may be, for example, a trained evaluating function.

In general, a trained function maps cognitive functions that humans associate with other human brains. Via training based upon training data (e.g., machine learning), the trained function is capable of adapting itself to new circumstances and of detecting and extrapolating patterns. Another expression for “trained function” is “trained machine learning model.”

In general, parameters of a trained function may be adapted via training. For example, supervised learning, semi-supervised learning, unsupervised learning, reinforcement learning, and/or active learning may be used. In addition, representation learning (also known as “feature learning”) may be used. The parameters of the trained function may be adapted, for example, iteratively via a plurality of training steps. For example, with the training, a particular cost function may be minimized. For example, during training of a neural network, the back propagation algorithm may be utilized.

A trained function may include, for example, a neural network, a support vector machine (SVM), a decision tree, and/or a Bayesian network, and/or the trained function may be based on k-means clustering, Q-learning, genetic algorithms, and/or assignment rules. For example, a neural network may be a deep neural network, a convolutional neural network (CNN), or a deep CNN. Further, the neural network may be an adversarial network, a deep adversarial network, and/or a generative adversarial network (GAN).

A convolutional neural network (CNN) is a neural network that uses a convolution operation rather than a general matrix multiplication in at least one of its layers, the convolutional layer. For example, a convolutional layer may perform a scalar product of one or more convolution kernels with the associated data/images of the convolutional layer, where the entries of the one or more convolution kernels are the parameters or weights that are adapted through training. For example, the Frobenius inner product and the ReLu activation function may be used. A CNN may include additional layers (e.g., pooling layers, fully connected layers, and normalization layers).

By way of CNNs, input images (e.g., scout images) may be processed extremely efficiently since a convolution operation based on different kernels may extract the most widely differing image features so that, by adapting the weights of the convolution kernel, the relevant image features may be found during the training. Further, based on the sharing of the weights in the convolutional layer kernels, fewer parameters have to be trained so that an overfitting in the training phase is avoided, and more rapid training or a larger number of layers are allowed in the CNN. The efficiency of the network is thus increased.

In a development, it may be provided that the trained evaluating function includes a region-based CNN (R-CNN). In an R-CNN, region proposals are generated, features extracted, and objects classified while their bounding boxes are refined. In this way, the object information may be determined in an extremely quick and robust manner. For example, a Faster R-CNN may be used that is based upon a ResNet50.

For training the trained evaluating function, both real and also simulated image recordings may be used. In order to determine simulated training data, for example, CBCT image datasets without metal objects may be utilized in which metal objects are then randomly distributed and projection images are simulated.

In a development of the present embodiment, it may be provided that at least one background information item is provided that describes the at least one metal object relevant to artifact formation (e.g., its object class), and that distinguishes it from other metal objects not to be taken into account. In the application of an evaluating function to at least one scout image, the evaluating function takes the background information item into account as input data and/or a plurality of evaluating functions relating to different metal objects (e.g., different object classes), of which one is selected for use based on the background information, and/or in the at least partial determination of the object information item from navigation data, the background information item is used for selecting navigation data that is to be used.

In this way, it is provided that the object information item relates exclusively to metal objects relevant to the formation of metal artifacts and/or their position in the three-dimensional image dataset and that further metal objects that are regarded as less and/or not relevant may be removed from observation. By this, for example, relatively small metal objects or metal objects situated remote from the actual region of interest (e.g., surgical instruments and/or materials) are no longer taken into account in the determination of the artifact-reducing recording trajectory, so that, for example, optimal image quality may be achieved where it is needed. In addition, in known methods that determine metal masks voxel by voxel, for example, detecting any metal without observing the objects as such, problems arising such as, for example, influences from actually irrelevant small objects and the prioritizing of false metal objects, may be avoided. The evaluating function therefore detects metal objects in a targeted manner and determines object information only for those metal objects that correspond to the previously selected metal objects relevant to artifact formation that are of interest. Accordingly, the navigation data used also relates to these metal objects. Therefore, the artifact measurement information item relates only to the metal objects relevant to artifact formation. For example, the metal objects relevant to artifact formation described by the background information item may be those metal objects of interest, of which the mapping is the goal of the imaging (e.g., implants, the seating of which is to be checked). Formulated differently, procedures from the prior art that operate with binary metal masks fit the recording trajectory to a scene taking account of all the metal voxels that may include irrelevant metal objects alongside the relevant objects (e.g., the metal objects relevant to artifact formation that are of interest); in the present case, only the metal objects of interest relevant to artifact formation that are defined via the background information item are modeled, and the other metal objects/voxels are rejected.

In specific example embodiments, an evaluating function that is trained for both a plurality of object classes (e.g., determining these), as well as to provide evaluating functions trained for different object classes in order then to select the necessary evaluating functions may be used.

Before an imaging procedure, for example, it may be determined (e.g., automatically) which metal objects of interest relevant to artifact formation are expected in the recording region, and a corresponding background information item may be passed on to the evaluating function as input data and/or used for selection of at least one suitable evaluating function and/or suitable navigation data. For example, the imaging goal from which the metal objects relevant to artifact formation may be determined may be derived automatically from information that has been acquired, for example, during a patient registration, is present in an electronic patient file and/or may be retrieved from a hospital information system/radiology information system (HIS/RIS) and/or a workflow management system. If, in an example, the position of pedicle screws is to be checked, the pedicle screws represent the metal objects relevant to artifact formation.

In the case of object-resolved artifact measurement information, in the corresponding representation, a further restriction of the metal objects relevant to artifact formation may take place as previously described based on a user input (e.g., only selected metal objects relevant to artifact formation being included in an optimization process).

In a specific embodiment, it may be provided that, for example, via the evaluating function, an object class and/or at least two marked out reference points of the metal object and/or at least one bounding box round the metal object is determined as object information for each metal object relevant to artifact formation. For example, the reference points and/or the bounding box may be selected so that the basic geometric form may be parameterized as simply as possible. In a specific example, it may be provided that the reference points in an elongate metal object relevant to artifact formation are at least two key points defining the length in the direction of greatest extent. For a pedicle screw, for example, a key point on the screw tip (e.g., end of the screw shaft) and a key point on the screw head may be provided along the longitudinal direction. If the pedicle screw is modeled as an ellipsoid, its main axis may be oriented in the head-to-tip direction defined by the key points and scaled according to the head-to-tip spacing. The second and third axes of the ellipsoid may be dimensioned according to a specification that defines the usual diameters of pedicle screws (e.g., 5 mm).

Suitably, each object class may be assigned to a basic form that is to be used. For different object classes, it may be useful to use different basic forms, as already indicated. Here, an assignment of basic forms to object classes that are anyway determined by the (e.g., trained) evaluating function is particularly simple. Thus, in a simple manner, a correct modeling adapted to the object class may take place.

In addition to the method, the present embodiments also relate to an X-ray facility having a control facility and a recording arrangement for recording two-dimensional projection images in different projection geometries along a recording trajectory, from which a three-dimensional image dataset may be reconstructed. The recording arrangement includes an X-ray radiator and an X-ray detector. The control facility is configured for carrying out a method according to the present embodiments. All the embodiments relating to the method may be transferred analogously to the X-ray facility according to the present embodiments, with which the above mentioned advantages may therefore also be achieved.

The control facility includes at least one processor and at least one storage device and may have functional units formed by hardware and/or software in order to perform acts of the method according to the present embodiments. For example, as known in principle, the control facility may have a recording unit in order to control the recording operation of the recording arrangement. The recording unit may be configured, for example, before the recording of projection images from which a three-dimensional image dataset is to be reconstructed, to record from a recording region that includes at least one metal object relevant to artifact formation, at least one two-dimensional scout image of the recording region (e.g., at least two scout images in different, mutually perpendicular projection geometries). Further, the recording unit may be configured to record the projection images along the artifact-reducing recording trajectory determined in the method according to the present embodiments. The control facility may further include an evaluating unit in order to determine the object information item. For this purpose, the evaluating unit may be configured to evaluate the at least one scout image using at least one, for example, trained evaluating function to determine the object information item that defines the position, orientation, and extent for each of the at least one metal object relevant to artifact formation in each scout image. Further, the evaluating unit may be configured to evaluate navigation data of a navigation system regarding the object information item. The control facility may further have a first determining unit for determining an object model that defines at least one three-dimensional basic geometric form that defines the metal object relevant to artifact formation and its position, size, and orientation in the recording region for each metal object relevant to artifact formation, from the object information item. A second determining unit may be provided in order to determine an artifact measurement information item that predicts the strength of metal artifacts that occur, for at least one of the at least one metal object relevant to artifact formation and at least one projection geometry (e.g., at least one potential recording trajectory), making use of at least one of the at least one object model. In order to use the artifact measurement information item in the determination of the artifact-reducing recording trajectory, a unit of use of the control facility may be utilized. The unit of use may include or be, for example, an optimizing unit that is configured to determine the artifact-reducing recording trajectory in an optimizing process minimizing a target function. The target function includes at least one artifact measurement term determined from the artifact measurement information item. Additionally or alternatively, the unit of use may include or be a representing unit that is configured for object-specific visualization of an expected artifact strength per metal object relevant to artifact formation in a representation that serves for user selection of an artifact-reducing recording trajectory to be used and/or for the user selection of at least one of the at least one metal object relevant to artifact formation. For further embodiments of the method, further functional units may also be provided, for example, an interface for receiving the background information item and/or a background information unit for its determination.

The X-ray facility may include a C-arm on which the X-ray radiator and the X-ray detector are arranged opposite one another. Via suitable degrees of freedom of movement of the C-arm and possibly of the X-ray radiator and/or the X-ray detector, the most varied of recording trajectories may be realized, from which the optimum radiator-detector recording trajectory may be selected, with regard to the artifacts, for metal objects relevant to artifact formation as the artifact-reducing recording trajectory. C-arm X-ray facilities may be utilized for medical interventions for monitoring them and checking the treatment success, including during the recording of three-dimensional CBCT image datasets. The X-ray facility may be part of an intervention workplace that, in addition to the X-ray facility, may also include a navigation system as described above.

A computer program according to the present embodiments is able to be loaded directly into a memory storage means (e.g., a memory storage device) of a control facility of an X-ray facility and has program means such that when the computer program is executed, the control facility is caused to carry out the acts of a method according to the present embodiments. The computer program may be stored on an electronically readable data carrier according to the present embodiments that therefore include control information stored thereon. The electronically readable data carrier includes the at least one computer program according to the present embodiments and is configured such that, when the data carrier is used in a control facility of a magnetic resonance facility, it causes the at least one computer program to carry out a method according to the embodiments. The data carrier may be, for example, a non-transient data carrier, such as, for example, a CD-ROM.

### BRIEF DESCRIPTION OF THE DRAWINGS

FIG. 1 shows a flow diagram of an example embodiment of a method;

FIG. 2 shows a schematic representation of a scout image;

FIG. 3 shows a schematic representation of a three-dimensional object model;

FIG. 4 shows a possible representation for a display of artifact measurement information;

FIG. 5 shows an X-ray facility according to an embodiment; and

FIG. 6 shows the functional structure of a control facility of the X-ray facility.

### DETAILED DESCRIPTION

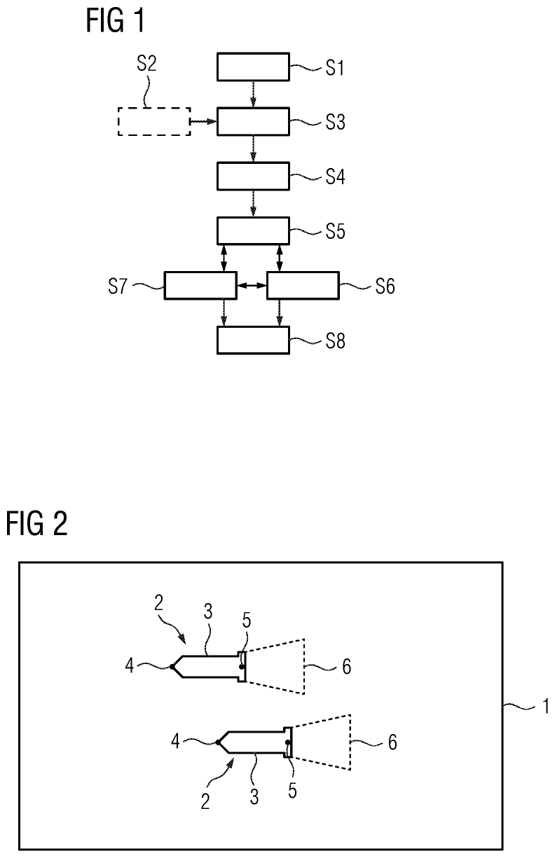

FIG. 1 shows a flow diagram of an example embodiment of a method. The method serves to determine an artifact-reducing recording trajectory to be used on a CBCT-capable X-ray facility on which a large number of projection geometries may be set, if metal objects are situated in a recording region. Along the recording trajectory, projection images are to be recorded making use of different projection geometries in order therefrom to reconstruct a three-dimensional image dataset of the recording region. The specific example relates to the checking of the correct positioning of metallic implants, wherein, in specific terms, and purely by way of example, pedicle screws are considered. Naturally, a large number of other application possibilities of the method according to the present embodiments are also open to persons skilled in the art.

In act S1, for this purpose, in this example embodiment, at least two (e.g., exactly two) scout images of the recording region are recorded. Different recording geometries are used, the central rays of which are perpendicular to one another. Metal objects situated in the recording region are acquired in this process. Embodiments may also be provided in which only one scout image is acquired or even no scout images are used, for example; rather, in a first act, navigation data of a navigation system that relates to metal objects in the recording region is received.

In an optional act S2 that may make use of scout images, although the method may be performed independently of the scout images, a background information item is determined and provided. The background information item defines which of the totality of the possibly existing metal objects are relevant to artifact formation, for example, in the form of at least one object class. This may be determined, for example, from a user input or automatically, for example, from an imaging goal present in an information system. In the present case, for example, pedicle screws are the metal objects relevant to artifact formation, since their placement is to be checked, and therefore, in their vicinity, a three-dimensional reconstructed image dataset should be as free of artifacts as possible.

In act S3, an evaluating function (e.g., trained) is applied to the scout images in order to determine object information for each object relevant to artifact formation. In the present case, a trained evaluating function is used, specifically for pedicle screws; more general evaluating functions may also be provided for a plurality of object classes. In example embodiments, it may be provided, based on the background information item, to select at least one suitable evaluating function as here.

In the present case, the trained evaluating function uses a “Faster R-CNN” architecture and delivers, as object information for each metal object relevant to artifact formation, a two-dimensional bounding box and herein, as reference points, two key points, one for the tip of the pedicle screw, one for the head. This is illustrated by FIG. 2 as a purely schematic representation of a scout image 1. This shows, by way of example, as metal objects 2 relevant to artifact formation, two pedicle screws 3. As reference points, the object information supplies key points 4 at the tips of the shaft and key points 5 at the screw heads. Further metal objects 6 not relevant to artifact formation including tulips and/or towers on the screw heads are indicated. These are not considered further in the determination of a suitable artifact-reducing recording trajectory of X-ray radiator and X-ray detector, so that an optimization focuses on the pedicle screws 3.

If navigation data is only or additionally used in other example embodiments, the background information item for the selection of relevant portions of the navigation data may be used, and act S4 may include its evaluation for determining the object information.

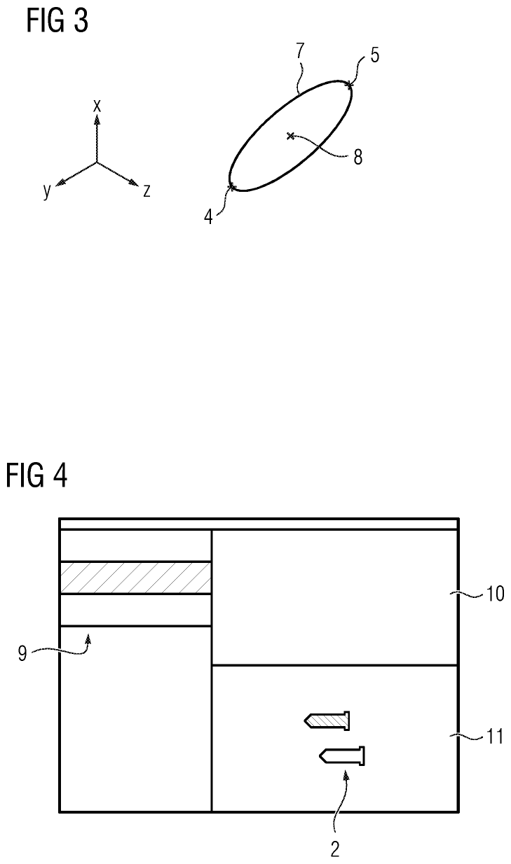

In act S4, based on the object information for each metal object relevant to artifact formation, a three-dimensional object model is determined based on a three-dimensional basic geometric form (e.g., thus, a geometric primitive). The pedicle screws 3 as elongate metal objects 2 relevant to artifact formation are mapped here (see FIG. 3) as ellipsoids 7, the longest main axis of which extends between the key points 4 and 5 of each pedicle screw that may be determined in three dimensions based on the plurality of scout images 1. The extent of the further axes of the ellipsoids 7 is selected according to typical dimensions of such pedicle screws 3, for example, as 5 mm. For better illustration, the ellipsoid in FIG. 3 is shown somewhat broader than it typically is in practice. Also shown in FIG. 3 is the geometric center 8 of the ellipsoid 7.

In act S5, the object models are used in order to determine an artifact measurement information item that is then used to determine the artifact-reducing recording trajectory. In the present case, a “total attenuation” approach is selected in which, as a measure for the contribution to the artifact strength, the maximum metal transirradiation length is used. For each projection geometry (e.g., characterized by, in the case of a C-arm, by two angles) that may be used along a recording trajectory, based on the object models, for example, object model-specifically and thus, for each metal object relevant to artifact formation (e.g., the artifact measurement information item), may be determined. While it is possible, for example, with metal objects relevant to artifact formation situated close to one another and/or having a complex arrangement, to render metal transirradiation length images significantly faster than when using a three-dimensional metal mask, it also enables the use of basic forms to determine them analytically for individual object models. Thus, with ellipsoids 7, the maximum metal transirradiation length may be simply determined analytically since the maximum metal transirradiation length always extends through the geometric center 8.

In the application case of pedicle screws 3, the pedicle screws 3 are mostly sufficiently separate spatially so that there is no risk of an overlap along the shanks; transversely thereto, it is less critical since with the smaller diameters, a reduction of the photon count below a critical level (e.g., “photon starvation”) does not arise. However, it may also be provided for an overlap also to be determined and taken into account computationally making use of an analytical relationship per object model.

In act S5, it is possible to determine a type of “metrics map” as artifact measurement information in that for conceivable/implementable projection geometries, the maximum metal transirradiation lengths are determined. In a C-arm and with projection geometries characterized by way of two angles, permitted combinations may be covered, for example, in 1° steps. If an artifact measurement information item is needed for a recording trajectory, the corresponding values may be retrieved from the metrics map. Naturally, it is also possible, however, always to determine the artifact measurement information item in a targeted manner for particular recording trajectories.

The artifact measurement information item may be used in determining the artifact-reducing recording trajectory in various specific ways, where the central types of use, which may also cooperate, are indicated schematically in FIG. 1 as acts S6, S7.

In act S6, an optimization process takes place making use of the artifact measurement information item. Therein, a target function is used that contains an artifact measurement term that is derived from the artifact measurement information item and is therefore aimed toward the minimization of the artifact strength for the metal objects 2 relevant to artifact formation. The target function may also have further terms, and/or the optimization process may, in general, use boundary conditions in order to map aspects such as the feasibility of the recording trajectories and suchlike. The optimization process may output a single artifact-reducing recording trajectory, but also a plurality of artifact-reducing recording trajectories that may be associated, for example, with different local minima that may result from different contributions from different metal objects relevant to artifact formation. In a variant that is simple to implement but is nevertheless suitable, the optimization may be targeted at the determination of at least one optimized tilt angle of the C-arm by which a circular path is also tilted as the recording trajectory. However, the artifact-reducing recording trajectory may also be determined using further degrees of freedom (e.g., those deviating from a circular path) for any desired paths and/or via a further term of the target function, paths selected such that a robust reconstruction remains possible.

Act S7 symbolizes a display of the artifact measurement information item in a representation that may serve for user selection of a suitable artifact-reducing recording trajectory, but may also permit the selection of particular (e.g., particularly interesting) metal objects 2 relevant to artifact formation for which optimization is then carried out anew. For example, in one embodiment, a tilt angle specified by a user for circular paths that may take place through interaction with the representation may always display the associated artifact measurement information, for example, resolved by metal objects relevant to artifact formation. In the case where a plurality of optimized recording trajectories are determined by an optimization process, the representation may also serve to select one therefrom.

For this purpose, FIG. 4 shows a representation with a plurality of optimized and recommended recording trajectories determined, for example, in an optimization process and that may be selected in a list 9. In a subwindow 10, all the artifact measurement information (e.g., for all the metal objects 2 relevant to artifact formation) is displayed to be comparable for all the recording trajectories. While in a subwindow 11, utilizing the determination resolved by metal objects 2, a representation image, for example, derived from or comprising a scout image 1 is displayed in which the metal objects 2 relevant to artifact formation are characterized (e.g., colored according to their share of the predicted metal artifact strengths). Thus, a user may select an artifact-reducing recording trajectory that, for metal objects 2 relevant to artifact formation of particular interest to him/her (e.g., newly inserted or last positioned pedicle screws 3) shows the lowest artifact strength.

In act S8, if an artifact-reducing recording trajectory that is to be used is determined, the recording may take place along the determined artifact-reducing recording trajectory.

FIG. 5 shows a schematic outline of an X-ray facility 12 according to the present embodiments. In the present case, the X-ray facility 12 includes a C-arm 13 on which an X-ray radiator 14 and an X-ray detector 15 are arranged opposite one another. The operation of the X-ray facility 12 is controlled by a control facility 16 (e.g., a controller) that is configured for carrying out the method according to the present embodiments. For example, the control facility 16 may drive actuators in order to actuate different projection geometries along a, for example, artifact-reducing recording trajectory by moving the C-arm 13.

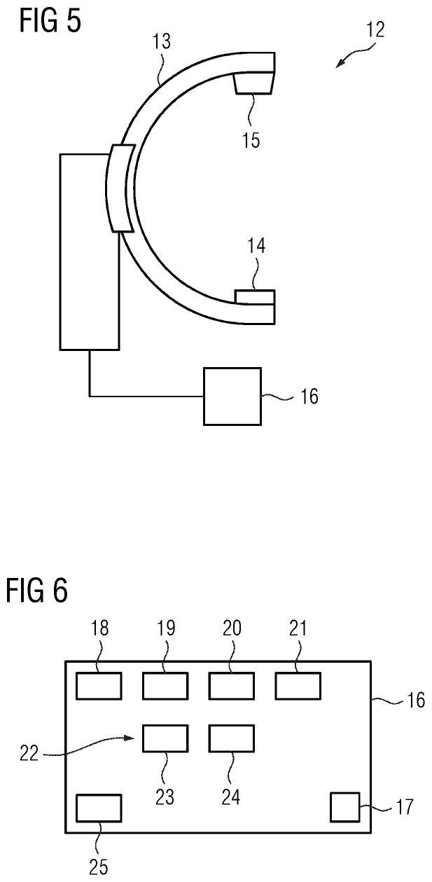

FIG. 6 shows the functional structure of the control facility 16 in greater detail. The control facility 16 includes at least one storage device 17 in which, for example, scout images 1, artifact measurement information, projection images, artifact-reducing recording trajectories, and suchlike may be at least temporarily stored. As is known in principle, the control facility 16 further includes a recording unit 18 in order to control the recording operation of the recording arrangement formed by the X-ray radiator 14 and the X-ray detector 15, and the projection geometry provided by the C-arm 13. The recording unit 18 is configured, before the recording of projection images from which the three-dimensional image dataset is to be reconstructed, to record the at least two two-dimensional scout images 1 of the recording region according to act S1. Further, the recording unit 18 is configured to record, according to act S8, the projection images along the artifact-reducing recording trajectory determined in the method according to the present embodiments.

In the present case, the control facility 16 further includes an evaluating unit 19 in order to evaluate the scout images 1 according to act S3 using the at least one trained evaluating function for determining the object information item. In addition, a first determining unit 20 is provided for determining the object models for each metal object 2 relevant to artifact formation from the object information according to act S4. A second determining unit 21 is configured to precalculate the artifact measurement information that defines the strength of metal artifacts that occur, for at least one of the at least one metal object 2 relevant to artifact formation and at least one projection geometry (e.g., at least one potential recording trajectory) according to act S5. In order to use the artifact measurement information item in the determination of the artifact-reducing recording trajectory, in the present case, two units of use 22 of the control facility 16 are shown. An optimizing unit 23 is configured for carrying out the optimizing process according to act S6. Additionally or alternatively, a representing unit 24 that serves for object-specific visualization of the expected artifact strength per metal object 2 relevant to artifact formation in a representation according to act S7 is provided. For the optional act S2, a background information unit 25 is also provided for determining the background information item.

The X-ray facility 12 may be part of an intervention workplace that may also include a navigation system, not described in detail here. Using the navigation system, metal objects relevant to artifact formation and/or instruments and/or materials used for their positioning may be tracked in a coordinate system registered to the coordinate system of the X-ray facility. Corresponding navigation data may be provided via an interface of the control facility 16.

Independent of the grammatical term usage, individuals with male, female or other gender identities are included within the term.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A computer-implemented method for operating an X-ray facility that comprises, for recording two-dimensional projection images in different projection geometries along a recording trajectory, from which a three-dimensional image dataset is reconstructable, a recording arrangement with an X-ray radiator and an X-ray detector, wherein before the recording of a recording region that comprises at least one metal object relevant to artifact formation, for determining an artifact-reducing recording trajectory, the method comprises:
determining an object information item that defines a three-dimensional position, orientation, and extent for each of the at least one metal object relevant to artifact formation;
determining, from the object information item for each of the at least one metal object relevant to artifact formation, an object model that defines at least one three-dimensional basic geometric form that defines the respective metal object relevant to artifact formation and a position, size and orientation of the artifact formation in the recording region; and
determining an artifact measurement information item that defines a strength of metal artifacts that occur, for one or more of the at least one metal object relevant to artifact formation and at least one projection geometry making use of one or more of the at least one object model,
wherein the artifact measurement information item is used in determining the artifact-reducing recording trajectory.

2. The computer-implemented method of claim 1, wherein:
the object information item is determined at least partially from navigation data of a navigation system for support during a preceding intervention for positioning the metal object relevant to artifact formation;
at least one two-dimensional scout image of the recording region is recorded and the at least one scout image is evaluated using at least one evaluating function to determine the object information item that defines at least the position, orientation, and extent for each of the at least one metal object relevant to artifact formation in each of the at least one scout image; or
a combination thereof.

3. The computer-implemented method of claim 2, wherein:
at least two two-dimensional scout images of the recording region are recorded in different projection geometries and evaluated, wherein the three-dimensional position, orientation, and extent for each of the at least one metal object relevant to artifact formation is determined at least partially from the scout image-related object information and the projection geometries of the scout images;
when a single scout image is used, at least one prior information item that defines the form and extent of the metal object is used for determining the three-dimensional position, orientation and extent of each of the at least one metal object relevant to artifact formation; or
a combination thereof.

4. The computer-implemented method of claim 1, wherein the artifact-reducing recording trajectory, at least one suggestion set of optimized recording trajectories that is offered to a user for selection, or a combination thereof is determined in an optimizing process minimizing a target function, and
wherein the target function comprises at least one artifact measurement term determined from the artifact measurement information item.

5. The computer-implemented method of claim 1, wherein the artifact measurement information item is determined as resolved by metal objects and is used for object-specific visualization of an expected artifact strength per metal object relevant to artifact formation in a representation that serves for user selection of an artifact-reducing recording trajectory to be used, for user selection of one or more of the at least one metal object relevant to artifact formation, or for a combination thereof.

6. The computer-implemented method of claim 1, wherein determining the artifact measurement information item comprises determining a metal transirradiation length for one or more of the at least one metal object relevant to artifact formation.

7. The computer-implemented method of claim 6, wherein:
determining the metal transirradiation length comprises rendering a transirradiation length image for the at least one projection geometry for one or more of the at least one object model;
determining the metal transirradiation length comprises using at least one analytical relationship based on a property of the basic geometric form; or
a combination thereof.

8. The computer-implemented method of claim 1, wherein an ellipsoid is used as a basic geometric form of the at least one three-dimensional basic geometric form.

9. The computer-implemented method of claim 1, wherein one or more of the at least one metal object relevant to artifact formation are modeled in the object model via a plurality of overlaid basic forms.

10. The computer-implemented method of claim 1, wherein:
at least one background information item that describes the at least one metal object relevant to artifact formation is provided, the at least one background information item distinguishing the at least one metal object relevant to artifact formation from other metal objects not to be taken into account, and when using an evaluating function for at least one scout image, the evaluating function takes the at least one background information item into account as input data, a plurality of evaluating functions is provided relating to different metal objects, of which one is selected for use based on the at least one background information item, or a combination thereof;
in the at least partial determination of the object information item from navigation data, the background information item is used for selecting navigation data that is to be used; or
a combination thereof.

11. The computer-implemented method of claim 10, wherein the at least one background information item is an object class of the at least one metal object relevant to artifact formation

12. The computer-implemented method of claim 10, wherein via the evaluating function, an object class, at least two marked out reference points, or a combination thereof of the at least one metal object, at least one bounding box around the at least one metal object is determined as object information for each of the at least one metal object relevant to artifact formation, or a combination thereof.

13. The computer-implemented method of claim 1, wherein the artifact-reducing recording trajectory is determined as a circular trajectory tilted by an optimized tilt angle.

14. An X-ray device comprising:
a controller; and
a recording arrangement for recording two-dimensional projection images in different projection geometries along a recording trajectory, from which a three-dimensional image dataset is reconstructable, wherein the recording arrangement comprises an X-ray radiator and an X-ray detector,
wherein the controller is configured for operating the X-ray device, wherein before the recording of a recording region that comprises at least one metal object relevant to artifact formation, for determining an artifact-reducing recording trajectory, the controller is further configured to:
determine an object information item that defines a three-dimensional position, orientation, and extent for each of the at least one metal object relevant to artifact formation;
determine, from the object information item for each of the at least one metal object relevant to artifact formation, an object model that defines at least one three-dimensional basic geometric form that defines the respective metal object relevant to artifact formation and a position, size and orientation of the artifact formation in the recording region; and
determine an artifact measurement information item that defines a strength of metal artifacts that occur, for one or more of the at least one metal object relevant to artifact formation and at least one projection geometry making use of one or more of the at least one object model, and
wherein the artifact measurement information item is used in determining the artifact-reducing recording trajectory.

15. In a non-transitory computer-readable storage medium that stores instructions executable by one or more processors for operating an X-ray facility that comprises, for recording two-dimensional projection images in different projection geometries along a recording trajectory, from which a three-dimensional image dataset is reconstructable, a recording arrangement with an X-ray radiator and an X-ray detector, wherein before the recording of a recording region that comprises at least one metal object relevant to artifact formation, for determining an artifact-reducing recording trajectory, the instructions comprise:
determining an object information item that defines a three-dimensional position, orientation, and extent for each of the at least one metal object relevant to artifact formation;
determining, from the object information item for each of the at least one metal object relevant to artifact formation, an object model that defines at least one three-dimensional basic geometric form that defines the respective metal object relevant to artifact formation and a position, size and orientation of the artifact formation in the recording region; and
determining an artifact measurement information item that defines a strength of metal artifacts that occur, for one or more of the at least one metal object relevant to artifact formation and at least one projection geometry making use of one or more of the at least one object model,
wherein the artifact measurement information item is used in determining the artifact-reducing recording trajectory.
